(19) United States
(12) Patent Application Publication
(10) Pub. No.: US 2018/0216075 A1
(43) Pub. Date: Aug. 2, 2018

(54) COMPOSITE BINDER MATERIAL, CELL AGGREGATE, CELL ACCUMULATION METHOD, CELL PRESERVATION METHOD AND METHOD OF MANUFACTURING COMPOSITE BINDER

(71) Applicant: FUJIFILM Corporation, Tokyo (JP)

(72) Inventors: Koju ITO, Kanagawa (JP); Shun GOTO, Kanagawa (JP); Souichi KOHASHI, Kanagawa (JP)

(73) Assignee: FUJIFILM Corporation, Tokyo (JP)

(21) Appl. No.: 15/939,300

(22) Filed: Mar. 29, 2018

Related U.S. Application Data
(63) Continuation of application No. PCT/JP2016/078418, filed on Sep. 27, 2016.

(30) Foreign Application Priority Data
Sep. 29, 2015 (JP) 2015-190568

(51) Int. Cl. C12N 5/071; C12M 3/00; C12M 1/12

(52) U.S. Cl. CPC C12N 5/0671; C12M 25/14; C12M 25/02; C12M 21/08

(57) ABSTRACT

Provided is a composite binder material, a cell aggregate, a cell accumulation method, a cell preservation method, and a method of manufacturing a composite binder that can supply oxygen and nutrients to cells.

A composite binder material comprises a binder material and a cell non-adhesive portion. A binder material formed in a membrane shape is provided between a first cell sheet and a second cell sheet. The cell non-adhesive portion formed at least in a portion of one membrane surface and/or other membrane surface of the binder material. A plurality of pores are formed in the binder material, and the pores open to the one membrane surface coming into contact with one sheet surface of the first cell sheet. The pores penetrate in a thickness direction and a surface direction along the one membrane surface. The porosity of the binder material is set to be at least 50%.

### CROSS-REFERENCE TO RELATED APPLICATIONS

This application is a Continuation of PCT International Application No. PCT/JP2016/078418 filed on 27 Sep. 2016, which claims priority under 35 U.S.C § 119(a) to Japanese Patent Application No. 2015-190568 filed on 29 Sep. 2015. The above application is hereby expressly incorporated by reference, in its entirety, into the present application.

### BACKGROUND OF THE INVENTION

### 1. Field of the Invention

The present invention relates to a composite binder material, a cell aggregate, a cell accumulation method, and a cell preservation method, and a method of manufacturing a composite binder.

### 2. Description of the Related Art

In order to realize regenerative medicine for three-dimensional organs such as the heart and the liver, a tissue body (hereinafter referred to as a three-dimensional cellular tissue body) formed of a so-called three-dimensionally formed cell group having sufficient thickness is indispensable. As a method for forming a three-dimensional cellular tissue body, there is a method for arranging a spherical cell aggregation called a spheroid in a planar shape, a method for overlapping a plurality of cell sheets which are sheet-like cell aggregations in a thickness direction, and a method for folding a cell sheet to be three-dimensional. The method for folding a cell sheet has an advantage that it is possible to relatively simply make a cell sheet three-dimensional as long as it is possible to make the cell sheet have a large area.

The cell sheet is usually produced in a culture vessel such as a petri dish, but it is necessary to take out the cell sheet from the culture vessel without any damage in order to handle the cell sheet in a case of lamination. Therefore, in WO2012/036224A (corresponding to US2013/171213 A1), for example, a method is used in which cell sheets are produced by culturing cells on culture supports of which the surfaces are coated with so-called temperature responsive polymers whose hydration force changes within a certain temperature range, the produced cell sheets are peeled off, and the plurality of cell sheets overlap each other. Specifically, cells are cultured in a temperature range where the hydration force of the temperature responsive polymer is weak, and are then cultured by changing the temperature to a temperature at which the hydration force of the temperature responsive polymer becomes strong to peel the cell in a sheet shape.

Even if a cell sheet is obtained without being damaged as described above, supply of oxygen and/or nutrients or the like from a culture solution in a medium to the cells is insufficient as the thickness of cell sheets laminated is increased, and therefore, there is a problem in that the cells die. Therefore, WO2012/036224A proposes a method for forming a vascular network between a plurality of overlapping cell sheets and within a cell sheet.

### SUMMARY OF THE INVENTION

The method for arranging spheroids in a planar shape has a limitation on the thickness of the obtained cell aggregation, which is not a level in which the cell aggregation is usable as a three-dimensional cellular tissue body. Even if it is intended to further cultivate the cells using spheroids arranged in a planar shape to increase the thickness of the cells, supply of oxygen and/or nutrients or the like from a culture solution in a medium to the cells is insufficient, and therefore, cells die. In addition, the method for forming a vascular network using a cell sheet as in WO2012/036224A has a problem that it is complicated and time-consuming, and therefore, a simple alternative method is desired.

Therefore, an object of the present invention is to provide a composite binder material, a cell aggregate, a cell accumulation method, a cell preservation method and a method of manufacturing a composite binder that can supply oxygen and nutrients to cells.

The composite binder material of the present invention has a binder material disposed between a first cell group and a second cell group and formed in a membrane shape and a cell non-adhesive portion formed at least in a portion of one membrane surface and/or other membrane surface of the binder material. The binder material has a honeycomb structure where a plurality of pores open to the one membrane surface are regularly arranged along the one membrane surface. Each of the plurality of pores penetrates in a thickness direction or the plurality of pores penetrate in a surface direction along the surface direction, and the binder material has a porosity of at least 50%, that is, greater than or equal to 50%.

The first cell group and the second cell group are preferably any one of a cell aggregation and a plurality of scattered cells. The cell aggregation is preferably a cell sheet in which a plurality of cells are aggregated into a sheet shape or a spheroid in which a plurality of cells are aggregated in a spherical shape.

It is preferable that the plurality of pores penetrate in the thickness direction and a thickness of the binder material is within a range of 1 μm to 50 μm.

The binder material is folded in a state where a first region and a second region of the one membrane surface face each other, and the first cell group may be disposed in a state of being caught in the binder material.

The cell aggregate of the present invention includes a first cell group, a second cell group, and the above-described composite binder material provided between the first cell group and the second cell group. In the cell aggregate, the binder material is folded in a state where the first region and the second region of the one membrane surface face each other, and the first cell group may be caught in the binder material.

The cell accumulation method of present invention has the following first step and second step. In the first step, a first cell group and the above-described composite binder material overlap each other in a state where the first cell group and the one membrane surface of the binder material or the cell non-adhesive portion formed at least in a portion of the one membrane surface come into contact with each other. In the second step, a second cell group and the composite binder material overlap each other in a state where the second cell group and the other membrane surface of the binder material or the cell non-adhesive portion formed at least in a portion of the other membrane surface come into contact with each other.

It is preferable that the first cell group is the above-described cell aggregation, and the thickness of the first cell group is at most 200 μm, that is, less than or equal to 200 μm. It is preferable that the second cell group is the above-described cell aggregation, the thickness of the second cell group is at most 200 μm, that is, less than or equal to 200 μm, and the plurality of pores of the binder material penetrate in the thickness direction. It is preferable that the binder material is folded in a state where a first region and a second region of the one membrane surface face each other, and the first cell group is disposed in a state of being caught in the binder material.

The cell preservation method of the present invention is a cell preservation method for preserving cells by overlapping a first cell group and a second cell group each other, the method including: overlapping the first cell group and the above-described composite binder material each other in a state where the first cell group and the one membrane surface of the binder material or the cell non-adhesive portion formed at least in a portion of the one membrane surface come into contact with each other; overlapping the second cell group and the composite binder material each other in a state where the second cell group and the other membrane surface of the binder material or the cell non-adhesive portion formed at least in a portion of the other membrane surface come into contact with each other; and holding the cell aggregate in which the first cell group and the second cell group overlap each other through the composite binder material in a state of being in contact with a culture solution of the cells.

The manufacturing method of the present invention is a method of manufacturing the above-described composite binder material, the method including: a first step of preparing a solution by dissolving a material to constitute the cell non-adhesive portion in a solvent; and a second step of coating the binder material with the solution and drying a coated membrane. It is preferable that in the second step the binder material is coated with the solution by dipping the binder material in the solution.

According to the present invention, it is possible to supply oxygen and nutrients to cells

### BRIEF DESCRIPTION OF THE DRAWINGS

FIG. 1 is a cell aggregate in which the present invention is embodied.

FIG. 2 is a plan view of a binder material.

FIG. 3 is a cross-sectional view taken along line III-III of FIG. 2.

FIG. 4 is a cross-sectional view taken along line IV-IV of FIG. 2.

FIG. 5 is an explanatory view of a method for manufacturing the cell aggregate.

FIG. 6 is an explanatory view showing a layer structure of another cell aggregate.

FIG. 7 is a schematic view showing another cell aggregate.

FIG. 8 is an explanatory view showing a layer structure of another cell sheet.

FIG. 9 is a schematic cross-sectional view of a composite binder material.

FIG. 10 is a schematic view showing another cell aggregate.

### DESCRIPTION OF THE PREFERRED EMBODIMENTS

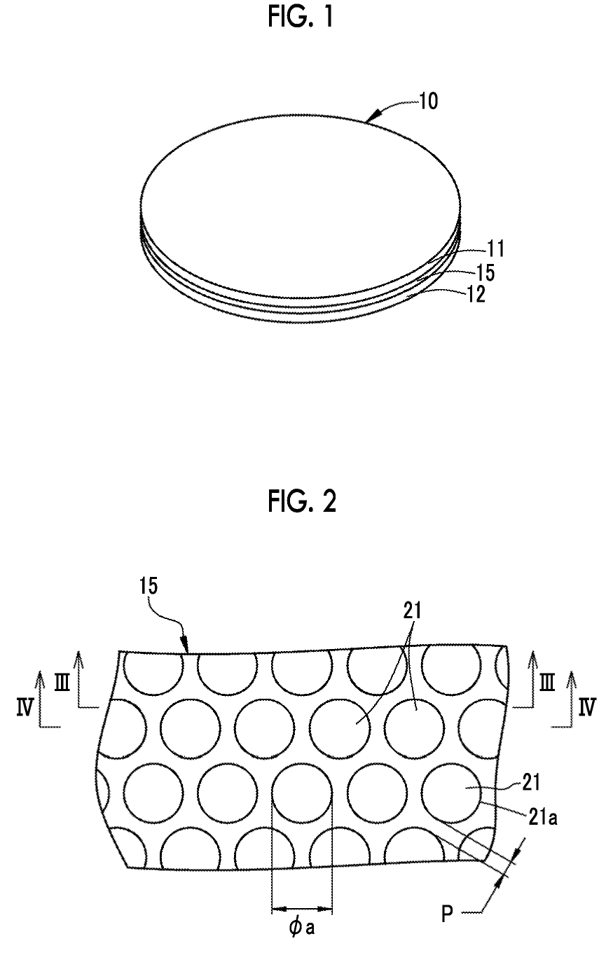

In FIG. 1, a cell aggregate 10 includes a first cell sheet 11, a second cell sheet 12, and a binder material 15 disposed between the first cell sheet 11 and the second cell sheet 12. The first cell sheet 11 and the second cell sheet 12 are cell aggregations in which a plurality of cells are aggregated in a sheet shape and include an extracellular matrix. A plurality of cells form a sheet shape through intervention of this extracellular matrix. The binder material 15 is for supplying oxygen and nutrients to the cells of the first cell sheet 11. In this example, the binder material 15 supplies oxygen and nutrients to both the cells of the first cell sheet 11 and the second cell sheet 12. The details of the binder material 15 will be described below using another drawing.

In this example, the first cell sheet 11, the second cell sheet 12, and the binder material 15 are in a circle having a diameter of 20 μm in a case where the first cell sheet, the second cell sheet, and the binder material are seen from above in FIG. 1, that is, in a case where the cell aggregate 10 is seen from a direction perpendicular to the surface of the first cell sheet 11 side. However, the diameters are not limited thereto. In addition, the first cell sheet, the second cell sheet, and the binder material may have a shape other than a circle, for example, an ellipse, a rectangle, a polygon, an irregular shape, or the like.

In this example, the thickness T11 of the first cell sheet 11 and the thickness T12 of the second cell sheet 12 are set to 100 μm, but the present invention is not limited thereto. However, the thickness T11 and the thickness T12 are preferably at least 50 μm from the viewpoint of ease of handling of the first cell sheet 11 and the second cell sheet 12, and at most 200 μm from the viewpoint of reliably supplying oxygen and nutrients to the cells of the first cell sheet 11 and the second cell sheet 12, that is, within a range of 50 μm to 200 μm. The thickness T11 and the thickness T12 are more preferably within a range of 60 μm to 180 μm, and are still more preferably within a range of 70 μm to 160 μm. In FIG. 1, the thickness T11, the thickness T12, and the thickness T15 of the binder material 15 which will be described below in detail are greatly exaggerated.

The cell types of the first cell sheet 11 and the second cell sheet 12 may be the same as or different from each other, and are the same as each other in the present embodiment. The cells in the present embodiment are hepatocytes. However, the present invention is not limited thereto, and the cells are, for example, respectively cells constituting a cardiac tissue, a liver tissue, a kidney tissue, an adrenal tissue, a skin tissue, and a mucosal tissue. In a case where plural types of cells coexist in each tissue, any one of the cells may be used, or two or more types of the cells may be used.

The cells of the first cell sheet 11 and the second cell sheet 12 are cultured cells obtained, for example, by culturing cells seeded in a culture medium. In this example, the first cell sheet 11 and the second cell sheet 12 contain a culture solution used for culturing. However, a new culture solution may be used instead of the culture solution used for the culturing, or a mixed solution thereof may be used.

The binder material 15 is for supplying oxygen and nutrients to the cells of the first cell sheet 11 and the second cell sheet 12 as described above. The binder material 15 is formed into a membrane shape. The binder material 15 overlaps with the first cell sheet 11 and the second cell sheet 12 each other in a state where one membrane surface (hereinafter, referred to as a first membrane surface) comes into contact with one sheet surface of the first cell sheet 11 and the other membrane surface (hereinafter, referred to as a second membrane surface) comes into contact with one sheet surface of the second cell sheet 12.

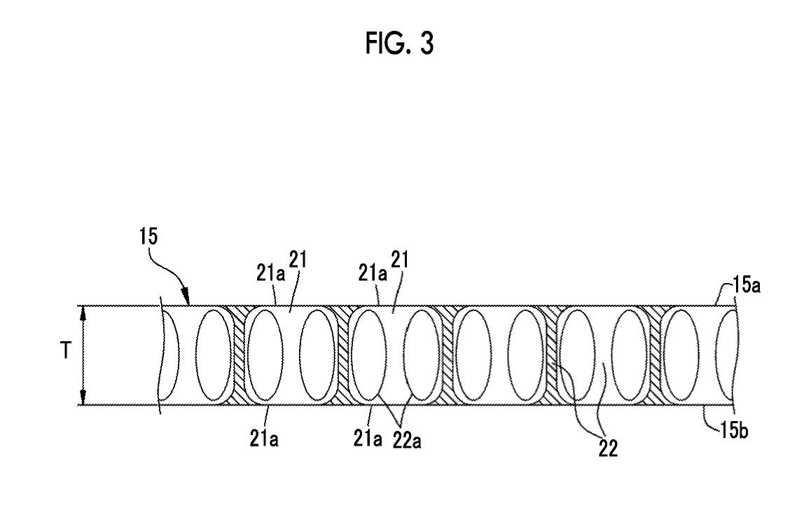

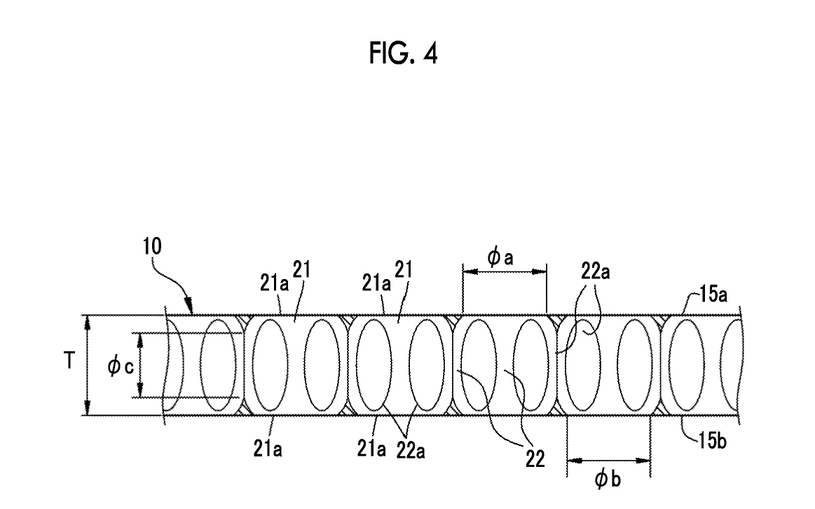

The binder material 15 will be described while referring to FIGS. 2 to 4. FIG. 2 shows the first membrane surface. In FIGS. 3 and 4, a reference numeral 15a is given to the first membrane surface and a reference numeral 15b is given to the second membrane surface.

The binder material 15 has a plurality of pores 21 open to the first membrane surface 15a. The pores 21 are for guiding oxygen and nutrients to the first cell sheet 11 (refer to FIG. 1) disposed on the first membrane surface 15a. In this example, the pores 21 also open to the second membrane surface 15b in addition to the first membrane surface 15a, and are for guiding oxygen and nutrients not only to the first cell sheet 11 but also to the second cell sheet 12 disposed on the second membrane surface 15b. The pores 21 penetrate the binder material 15 in a direction perpendicular to the first membrane surface 15a and the second membrane surface 15b, that is, in the thickness direction, and open to the first membrane surface 15a and the second membrane surface 15b to form an opening part (hereinafter, referred to as a surface opening part) 21a.

The plurality of pores 21 are regularly arranged along the first membrane surface 15a, in this example, more specifically, in a matrix shape. The size and the shape of each pore 21 are constant, and the size and the shape of each surface opening part 21a are also constant. In such a binder material 15, the pores 21 are densely arranged in a state where six surrounding pores 21 are arranged at each apex of a hexagon around an arbitrary pore 21 in a case where the binder material is seen from a direction perpendicular to the first membrane surface 15a. As a result, the binder material 15 has a honeycomb structure formed into a honeycomb shape.

In the honeycomb structure, the shape of the surface opening part 21a and the shapes of cross sections of the pores 21 parallel to the first membrane surface 15a are not necessarily hexagonal. In this example, the shape of the surface opening part 21a is circular. In some cases, there is a case where the shapes of the surface opening part 21a and the pores 21 in the cross section parallel to the first membrane surface 15a are, for example, a substantially rounded hexagonal shape, a substantially rounded octagonal shape, or the like, depending on the density of pores 21 of the first membrane surface 15a per unit area, the distance between adjacent pores 21, or the like, and the honeycomb structure includes such an embodiment. In addition, the honeycomb structure also includes a structure in which adjacent pores 21 are connected to each other in the binder material 15 as in this example in addition to a structure in which the pores 21 are independent from each other. Furthermore, the arrangement of the pores 21 is not limited to those described above. Three to five pores or seven or more pores 21 may be arranged around an arbitrary pore 21, and the pores 21 may be squarely arranged.

In FIGS. 3 and 4, the thickness of partition wall 22 between adjacent pores 21 is drawn exaggeratedly with respect to a thickness T15 of the binder material 15. In this example, the thickness of the partition wall 22 separating adjacent pores 21 from each other gradually decreases from each of the first membrane surface 15a and the second membrane surface 15b toward the center in the thickness direction.

In this example, the partition wall 22 has a partition opening part 22a formed substantially at the center in the thickness direction. Accordingly, adjacent pores 21 are connected to each other in the binder material 15 in the direction along the first membrane surface 15a (hereinafter, referred to as a surface direction). However, there is also a case where the partition wall opening part 22a is not formed in the partition wall 22, and in this case, the pores 21 are individually independent. The partition opening part 22a is for guiding oxygen and nutrients to each pore 21.

From the viewpoint of guiding oxygen and nutrients to the first cell sheet 11, the pores 21 may penetrate any one of the thickness direction and the surface direction. However, from the viewpoint of further reliably guiding oxygen and nutrients to the first cell sheet 11 and/or guiding oxygen and nutrients to the second cell sheet 12 in addition to the first cell sheet 11, the pores preferably penetrate in both the thickness direction and the surface direction as in the present embodiment.

The diameter of the surface opening part 21a, that is, the opening diameter ϕa of a pore 21 on the first membrane surface 15a is set to be within a range of 2 μm to 100 μm, and is 20 μm in the present embodiment. The opening diameter ϕa is more preferably within a range of 5 μm to 70 μm, and still more preferably within a range of 10 μm to 50 μm. In a case where a pore such as a pore 21 penetrating in the thickness direction is formed, it is preferable that the opening diameter ϕb of the pore on the second membrane surface 15b is similarly within the range of 2 μm to 100 μm, and is 20 μm in the present embodiment.

In addition, the opening diameter ϕc of the partition opening part 22a is preferably within the range of 2 μm to 80 μm.

The porosity of the binder material 15 is set to be at least 50%, that is, greater than or equal to 50%. This porosity is calculated using (V2/V1)×100, in a case where the volume in a case where the pores 21 are not formed in the binder material 15 is set to V1 and the sum of the volumes of the plurality of pores 21 is set to V2. The volume V2 can be calculated from the maximum diameter, the opening diameter ϕa, and the opening diameter ϕb of a pore 21 in the inside of the binder material 15, using an image taken by a scanning electron microscope (SEM). The porosity of the binder material 15 is preferably within a range of 50% to 90%.

The distance (hereinafter referred to as an opening part pitch) P between adjacent surface opening parts 21a in the first membrane surface 15a is not particularly limited. However, the distance is preferably within a range of 3 μm to 120 μm, and is 24 μm in the present embodiment. The opening part pitch P is more preferably within a range of 5 μm to 100 μm, and still more preferably within a range of 10 μm to 60 μm. The opening diameter ϕa and the opening part pitch P are obtained, for example, from an image of SEM.

In a case where the pores 21 penetrate in the thickness direction, the thickness T15 of the binder material 15 is preferably within a range of 1 μm to 50 μm, and is 10 μm in the present embodiment. The thickness T15 in the case where the pores 21 penetrate in the thickness direction is more preferably within a range of 1 μm to 50 μm, and still more preferably within a range of 2 μm to 40 μm. In a case where the pores 21 penetrate only in the surface direction, the thickness T15 is not particularly limited.

The binder material 15 is formed of a hydrophobic polymer in this example. Polylactic acid, polycaprolactone, polyglycolic acid, polydioxanone, polyhydroxybutyrate, polybutadiene, polyurethane, polystyrene (PS), polymethyl methacrylate, and polycarbonate, and copolymers containing repeating units thereof are preferable as the hydrophobic polymer, and polylactic acid is used in the present embodiment. Polylactic acid, polycaprolactone, polyglycolic acid, and copolymers thereof are particularly preferable among the above-described polymers from the viewpoint of imparting a decomposition or absorptive function over time due to in vivo use. In addition, polycaprolactone and polybutadiene having elasticity are particularly preferable from the viewpoint of being applied to a folded portion or an elastic portion, for example, being attached to a folded portion or an elastic portion.

The binder material 15 may contain, for example, an amphiphilic compound in addition to the hydrophobic polymer, and the amphiphilic compound may be any of a polymer, an oligomer, and a monomer. The mass of the amphiphilic compound in the case where the amphiphilic compound is contained is preferably lower than or equal to 10 with respect to mass of 100 of the binder material 15.

The operation of the above-described configuration will be described. The first cell sheet 11 is disposed in a state where one sheet surface comes into contact with the first membrane surface 15a of the binder material 15 and the second cell sheet 12 is disposed in a state where one sheet surface comes into contact with the second membrane surface 15b of the binder material 15. The pores 21 of the binder material 15 open to the first membrane surface 15a and penetrate in the thickness direction, and therefore, in a case where the second cell sheet 12 comes into contact with a culture solution, the culture solution is guided from the second cell sheet 12 to the pores 21 of the binder material 15, and is then guided to the first cell sheet 11 through the pores 21. Since the culture solution contains nutrients and dissolved oxygen, nutrients and oxygen are supplied to the cells of the first cell sheet 11. In addition, in a case where the other sheet surface 11b of the first cell sheet 11 is exposed to the outside, oxygen in the external atmosphere is also guided to the first cell sheet 11. In this case, oxygen in the external atmosphere is guided from the first cell sheet 11 to the binder material 15, and is then guided to the second cell sheet 12 through the pores 21 of the binder material 15. Accordingly, oxygen is supplied to the cells of second cell sheet 12.

Since the pores 21 of the binder material 15 penetrate in the thickness direction as described above, any one of the first cell sheet 11 and the second cell sheet 12 comes into contact with oxygen, so that oxygen is guided to the other, and therefore, is supplied to the cells of the first cell sheet 11 and second cell sheet 12. Likewise for nutrients, any one of the first cell sheet 11 and the second cell sheet 12 comes into contact with the culture solution, so that nutrients of the culture solution are guided to the other cell sheet and supplied to the other cells. By this supply, the cells are preserved in a viable state. Similar guidance is applied to waste products. Therefore, the cells are preserved in a state where the survival of the cells is more reliably maintained from the viewpoint of metabolism.

Since the pores 21 penetrate in the surface direction, oxygen or nutrients taken in from, for example, side edges of the binder material 15 are guided to the each of the pores 21 through the partition opening part 22a. Since each of the pores 21 is open to the first membrane surface 15a, oxygen and nutrients which have been guided into the pores 21 face the first cell sheet 11. Accordingly, oxygen and nutrients are supplied to the cells of first cell sheet 11. In addition, since each of the pores 21 also opens to the second membrane surface 15b in this example, oxygen and nutrients are similarly guided to the second cell sheet 12, and therefore, oxygen and nutrients are supplied also to the second cell sheet 12.

Since the pores 21 in this example penetrate in both the thickness direction and the surface direction, oxygen and nutrients taken into the binder material 15 are more easily diffused into the entirety of the inside of the binder material 15. Accordingly, oxygen and nutrients are more reliably guided to both the first cell sheet 11 and the second cell sheet 12 to be more reliably supplied to both cells. As described above, the binder material 15 functions as a cell preservation material for preserving cells in a viable state.

Since the opening diameter ϕa is set to be greater than or equal to 2 μm, oxygen and nutrients, in particular, nutrients containing molecules larger than oxygen, are reliably guided to the first cell sheet 11. Since the opening diameter ϕa of the first membrane surface 15a is set to be less than or equal to 100 μm, the strength in a case where the binder material 15 supports the first cell sheet 11 is sufficient. Since the opening diameter ϕb is similarly set to be within the range of 2 μm to 100 μm, oxygen and nutrients, in particular, nutrients are reliably guided to the second cell sheet 12, and the effect for preserving cells is enhanced.

Since the opening diameter ϕc is greater than or equal to 2 μm, oxygen and nutrients, in particular, nutrients are more sufficiently guided to the first cell sheet 11 as compared to a case where the opening diameter ϕc is less than 2 μm. Since the opening diameter ϕc is less than or equal to 80 μm, it is more advantageous in terms of durability against compression of the binder material 15 as compared to a case where the opening diameter ϕc is larger than 80 μm.

Since the porosity of the binder material 15 is greater than or equal to 50%, oxygen and nutrients are reliably guided to the cells in the entire sheet surface region of the first cell sheet 11 and the second cell sheet 12.

Since the opening part pitch P is greater than or equal to 3 μm, the strength of the binder material 15 increases as compared to a case where the opening part pitch P is smaller than 3 μm. Since the opening part pitch P is less than or equal to 120 μm, oxygen and nutrients are more reliably guided to the cells on the entire sheet surface of the first cell sheet 11 and the second cell sheet 12 as compared to a case where the opening part pitch P is larger than 120 μm.

The cell group tends to change the posture so as to achieve a stable state. Likewise, the cell group tends to change the posture so as to be in a stabler state during culturing for proliferation. Accordingly, the cells of the first cell sheet 11 and the second cell sheet 12 mutually achieve proliferation, migration, and self-organization using gaps of the pores 21 in a case where these pores 21 penetrate in the thickness direction. Accordingly, after a certain period of time, in some cases, cultured cells are newly formed from each of the cells of the first cell sheet 11 and the second cell sheet 12 and integrated in the binder material 15. In the present embodiment in which the pores 21 penetrate in the thickness direction, the thickness T15 of the binder material 15 is set to be less than or equal to 50 μm. Therefore, it becomes easy for the cells of the first cell sheet 11 and the second cell sheet 12 to mutually recognize the existence of each other as compared to a case where the thickness T15 is larger than 50 μm, and the above-described integration easily occurs. In addition, since the thickness T15 is greater than or equal to 1 μm, this case is more excellent in terms of the strength as the cell aggregate 10 as compared to a case where the thickness T15 is less than 1 μm.

The effect of the supply of oxygen and nutrients to the cells of the first cell sheet 11 using the binder material 15 is larger as the thickness of the first cell sheet 11 is large. The effect becomes particularly remarkable in a case where the thickness of the first cell sheet 11 is greater than or equal to 50 μm. Likewise, the effect of the supply of oxygen and nutrients to the cells of the second cell sheet using the binder material 15 is larger as the thickness of the second cell sheet 12 is large. The effect becomes particularly remarkable in a case where the thickness of the second cell sheet 12 is greater than or equal to 50 μm.

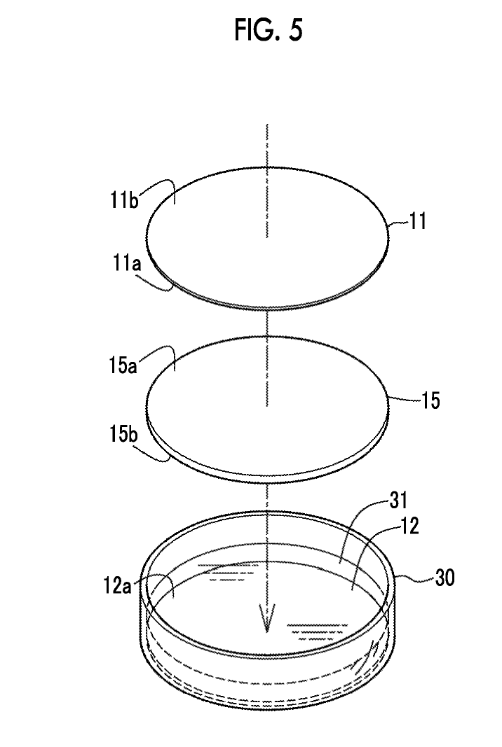

A method for manufacturing the cell aggregate 10 will be described while referring to FIG. 5. The method for manufacturing the cell aggregate 10 has a first step of overlapping the first cell sheet 11 and the binder material 15 each other and a second step of overlapping the second cell sheet 12 and the binder material 15 each other. Examples are as follows.

As described above, the first cell sheet 11 and the second cell sheet 12 are respectively formed from cultured cells. In a culture vessel 30 in which the second cell sheet 12 is formed, the second cell sheet 12 is in a state of being accommodated in a bottom portion, and a culture solution 31 which has been used for culturing is also accommodated in the culture vessel 30. The first cell sheet 11 is also formed using a culture vessel (not shown in the drawing) similar to the culture vessel 30 and is taken out of the culture vessel. The binder material 15 is disposed on the second cell sheet 12 in the culture vessel 30 in a posture in which the second membrane surface 15b faces downward in FIG. 5. Accordingly, the binder material 15 and the second cell sheet 12 overlap each other in a state where the second membrane surface 15b of the binder material 15 is brought into contact with one sheet surface 12a of the second cell sheet 12. The first cell sheet 11 is disposed on the binder material 15. Accordingly, the binder material 15 and the first cell sheet 11 overlap each other in a state where one sheet surface 11a of the first cell sheet 11 is brought into contact with the first membrane surface 15a of the binder material 15.

In the example of the above-described manufacturing method, the cell aggregate 10 is manufactured in the order of a second step and a first step. However, the cell aggregate 10 may be manufactured in the order of the first step and the second step such that after the first cell sheet 11 and the binder material 15 overlap each other, the binder material 15 overlapping the first cell sheet 11 overlaps the second cell sheet 12 each other.

In addition, in the example of the above-described manufacturing method, the binder material 15 and the first cell sheet 11 are sequentially stacked on the second cell sheet 12. However, instead of this order, the binder material 15 and the second cell sheet 12 may be sequentially stacked on the first cell sheet 11.

In the method for preserving the cells of the first cell sheet 11 and the second cell sheet 12, the cell aggregate 10 manufactured through the above-described manufacturing method is held in a state where the cell aggregate is brought into contact with the culture solution 31. According to the example of the above-described manufacturing method, at least the second cell sheet 12 among the first cell sheet 11, the second cell sheet 12, and the binder material 15 is brought into contact with the culture solution 31. Since the pores 21 penetrate the binder material 15 in the thickness direction, the culture solution 31 of the second cell sheet 12 passes through the binder material 15 and is guided to the first cell sheet 11. Accordingly, nutrients and dissolved oxygen in the culture solution 31 are supplied to the cells of first cell sheet 11. In addition, in a case where the other sheet surface 11b of the first cell sheet 11, in this example, the upper surface in FIG. 5 is located higher than the liquid surface of the culture solution 31, oxygen is also supplied from the external atmosphere to the cells of the first cell sheet 11. In this case, oxygen is also guided to the second cell sheet 12 through the pores 21 penetrating in the thickness direction.

In a case where the liquid surface of the culture solution 31 is higher than the second membrane surface 15b of the binder material 15, the culture solution 31 is taken into the binder material 15 from side edges of the binder material 15. Since the binder material 15 has the pores 21 penetrating in the surface direction and the pores 21 open to the first membrane surface 15a, the culture solution 31 is guided to the first cell sheet 11 through the pores 21. In addition, since the pores 21 of the binder material 15 also penetrate in the thickness direction, even if the thickness of the second cell sheet 12 is large, the culture solution 31 is guided from one sheet surface 12a coming into contact with the binder material 15 through the pores 21.

In cases where the cell aggregate 10 was manufactured by sequentially overlapping the binder material 15 and the second cell sheet 12 on the first cell sheet 11 in the culture vessel, and the pores 21 of the binder material 15 penetrate only in the surface direction without penetrating in the thickness direction, the manufactured cell aggregate 10 is held in the vessel containing the culture solution in a posture that the second cell sheet 12 is the lowermost layer.

The above-described example is an embodiment in which two cell sheets overlap each other, but the number of overlapping cell sheets is not limited to 2, and may be greater than or equal to 3. However, an embodiment is preferable in which the binder material 15 is provided between a first accumulation portion and a second accumulation portion so that the total thickness of a plurality of cell sheets (referred to as accumulation portions) continuously overlapping is less than or equal to 200 μm. Examples are as follows.

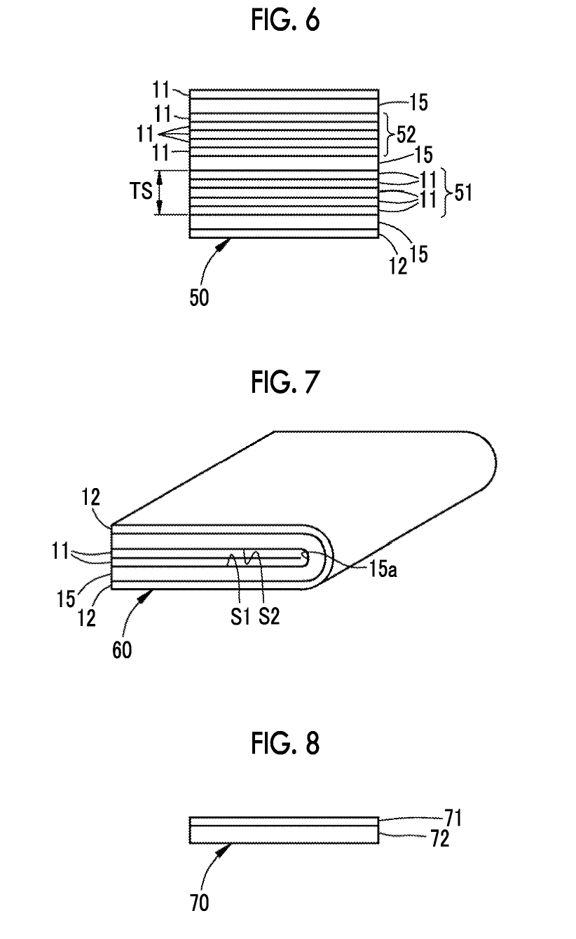

In FIG. 6, a cell aggregate 50 has a configuration in which a second cell sheet 12, a first accumulation portion 51 in which first cell sheets 11 continuously overlap each other, a binder material 15, a second accumulation portion 52 in which first cell sheets 11 continuously overlap each other, a binder material 15, and a first cell sheet 11 overlap in this order from the bottom in FIG. 6. The number of the first cell sheets 11 in each of the first accumulation portion 51 and the second accumulation portion 52 is set to five. It is preferable that the thickness TS of each of the first accumulation portion 51 and the second accumulation portion 52 is at most 200 μm, that is, less than or equal to 200 μm. In the present embodiment, each thickness TS is 100 μm. Each of the binder materials 15 is disposed between the second cell sheet 12 and the first accumulation portion 51, between the first accumulation portion 51 and the second accumulation portion 52, and between the second accumulation portion 52 and the uppermost first cell sheet 11 in FIG. 6. By providing the binder material 15 in an embodiment in which the thickness of each of the first accumulation portion 51 and the second accumulation portion 52 is suppressed to less than or equal to 200 μm in this manner, oxygen and nutrients are reliably guided to each cell sheet to be supplied to the cells of each cell sheet.

In this example, the number of the first cell sheets 11 in the first accumulation portion 51 and the second accumulation portion 52 is set to five, but is not limited to five. In addition, the first accumulation portion 51 and the second accumulation portion 52 are formed of the first cell sheets 11, but they may be formed of cell sheets different from each other.

The binder material 15 can be folded when in use. A cell aggregate 60 shown in FIG. 7 includes a binder material 15, a first cell sheet 11, and a second cell sheet 12. Also in FIG. 7, the thickness of the binder material 15, the first cell sheet 11, and the second cell sheet 12 is greatly exaggerated. The binder material 15, the first cell sheet 11, and the second cell sheet 12 in this example are the same as those in FIG. 1 except that they are formed in a rectangular shape, and therefore, the same reference numerals are used. Also in this example, the binder material 15, the first cell sheet 11, and the second cell sheet 12 may have other shapes such as a circle.

The binder material 15 is folded toward the first cell sheet 11 side in a state where a first region S1 and a second region S2 of the first membrane surface 15a face each other. In this example, the binder material 15 is folded after the first cell sheet 11 is disposed on the first membrane surface 15a. Therefore, the first cell sheet 11 is also folded in an overlapping manner, and is caught in the binder material 15. The first cell sheet 11 may be disposed between the first region S1 and the second region S2 after the binder material 15 is folded. The number of cell sheets overlapping between the first region S1 and the second region S2 may be greater than or equal to two. However, the thickness in the overlapping state is preferably at most 200 μm, that is, less than or equal to 200 μm. In this example, the number of times of folding the binder material 15 is set to one, but may be set to be greater than or equal to two.

The binder material 15 may be folded so that the first region and the second region of the second membrane surface 15b face each other. In this case, the pores 21 preferably penetrate in the thickness direction of binder material 15.

The binder material 15 may be wound in a roll shape when in use. A cell aggregate (not shown in the drawing) in the case where the cell aggregate is wound in a roll shape includes a binder material 15, a first cell sheet 11, and a second cell sheet 12. The binder material 15 is wound by having the first cell sheet 11 side inside in a state where the first region and the second region of the first membrane surface 15a overlap each other in a mutually identical direction. In this example, the binder material 15 is wound in a roll shape after the first cell sheet 11 is disposed on the first membrane surface 15a. Accordingly, the first cell sheet 11 is also wound and enters an overlapping state through the binder material 15 to be caught in the binder material 15. The number of cell sheets overlapping between the first region and the second region may be greater than or equal to two. However, the thickness in the overlapping state is preferably at most 200 μm, that is, less than or equal to 200 μm. In the binder material 15 in the case where the binder material is wound in a roll shape, the pores 21 preferably penetrate in the thickness direction of binder material 15. The number of times wound in a roll may be greater than or equal to two. The binder material 15 may be wound by having the second cell sheet 12 side inside in a state where the first region and the second region of the second membrane surface 15b overlap each other in a mutually identical direction. Even in this case, the pores 21 preferably penetrate in the thickness direction of the binder material 15.

The cell aggregates 10 and 50 in FIG. 1 and FIG. 6 may include a cell sheet including a support instead of the first cell sheet 11 and the second cell sheet 12. For example, a third cell sheet 70 shown in FIG. 8 includes a cell layer 71 and a support 72. Similarly to the first cell sheet 11 and the second cell sheet 12 described above, the cell layer 71 includes a plurality of cells and a cell matrix, and the cell group forms a sheet shape. Similarly to the first cell sheet 11 and the second cell sheet 12, the cell layer 71 may contain other substances such as a culture solution.

The support 72 is for supporting the cell layer 71 in a state where the cell layer is held in a sheet shape, and has a sheet shape. Similarly to the binder material 15, a plurality of pores (not shown in the drawing) which penetrate in the thickness direction and the surface direction and open to the surface coming into contact with the cell layer 71 are formed in the support 72.

In a case where this third cell sheet 70 is used instead of the first cell sheet 11 of the cell aggregate 10 described above, the third cell sheet 70 and the binder material 15 overlap each other in a state where the support 72 and the binder material 15 come into contact with each other. In a case where a support in which pores which penetrate only in the surface direction without penetrating in the thickness direction, and open to the surface coming into contact with the cell layer 71 are formed is used as the support 72, the third cell sheet 70 and the binder material 15 overlap each other in a state where the cell layer 71 and the binder material 15 are brought into contact with each other.

In a case where this third cell sheet 70 is used instead of the second cell sheet 12 of the cell aggregate 10 described above, the third cell sheet 70 and the binder material 15 overlap each other in a state where the binder material 15 and any one of the support 72 and the cell layer 71 come into contact with each other. Likewise in the case where a support in which pores which penetrate only in the surface direction without penetrating in the thickness direction, and open to the surface coming into contact with the cell layer 71 are formed is used as the support 72, the third cell sheet 70 and the binder material 15 overlap each other in a state where the binder material 15 and any one of the support 72 and the cell layer 71 are brought into contact with each other. Similarly to the thickness T11 of the first cell sheet 11 and the thickness T12 of the second cell sheet 12, it is preferable that the thickness of the third cell sheet 70 is at least 50 μm from the viewpoint of ease of handling, and at most 200 μm from the viewpoint of reliably supplying oxygen and nutrients to the cells of the cell layer 71 of the third cell sheet 70, that is, within a range of 50 μm to 200 μm.

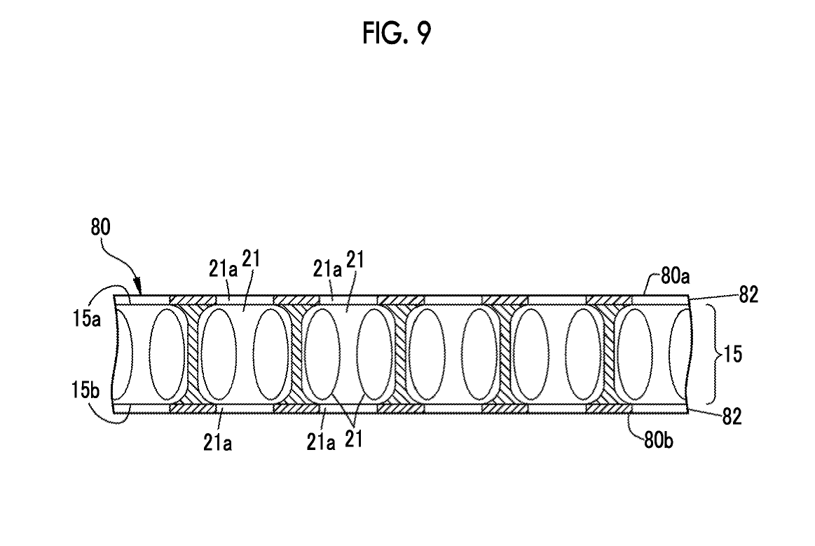

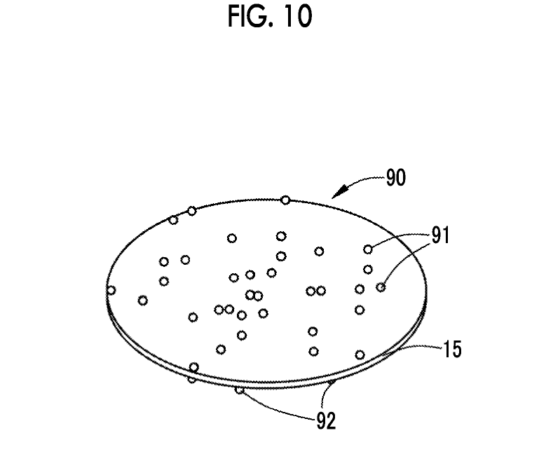

A composite binder material 80 shown in FIG. 9 may be used instead of the binder material 15. The composite binder material 80 includes a binder material 15 and a cell non-adhesive portion 82. The cell non-adhesive portion 82 is for suppressing the adhesion of the cells of the first cell sheet 11 and the second cell sheet 12 to the binder material 15 for a certain period of time.

The cell non-adhesive portion 82 is formed on each of the first membrane surface 15a and the second membrane surface 15b of the binder material 15, and forms surfaces 80a and 80b of the composite binder material 80, but is not formed in a surface opening part 21a. In this example, the cell non-adhesive portion 82 covers the first membrane surface 15a and the second membrane surface 15b except for the surface opening part 21a, but may have an embodiment in which the membrane surfaces are partially covered. The cell non-adhesive layer 82 may be provided only on any one of the first membrane surface 15a and the second membrane surface 15b. In addition, in this example, the cell non-adhesive layer 82 is not provided on the surface of a partition wall between pores 21, but may be provided on the surface of the partition wall. The cell non-adhesive portion 82 can be formed, for example, through a well-known dip-coating method. For example, the cell non-adhesive portion 82 can be formed such that the material constituting the cell non-adhesive portion 82 is dissolved in a solvent to prepare a solution, the binder material 15 is dipped in this solution, the solution is applied to the binder material 15, and then, the coated membrane is dried. Since the cell non-adhesive portion 82 is formed, the integration of the cells of the first cell sheet 11 and the second cell sheet 12 with the binder material 15 is suppressed for a certain period of time. Therefore, the cells are more reliably preserved due to the flow path of oxygen and nutrients which has been secured.

Polyethylene glycol (PEG) or a 2-methacryloyloxyethyl phosphorylcholine (MPC) polymer is preferable as the material constituting the cell non-adhesive layer 82.

The above-described example is a case where the cell aggregation is a cell sheet. However, the cell aggregation is not limited to the cell sheet, and may be a spheroid in which a plurality of cells are aggregated in a spherical shape. For example, the cell aggregate 90 shown in FIG. 10 includes a plurality of first spheroids 91, a plurality of second spheroids 92, and a binder material 15 disposed between the first spheroids 91 and the second spheroids 92. The binder material 15 is the same as that in the cell aggregate 10. The first cell spheroids 91 and the second cell spheroids 92 are cell aggregations in which a plurality of cells are aggregated in a spherical shape and include a cell matrix. A plurality of cells form a spherical shape through intervention of this extracellular matrix. The first spheroids 91 are scattered on the first membrane surface 15a of the binder material 15. The second spheroids 92 are scattered on the first membrane surface 15b of the binder material 15. The binder material 15 is for supplying oxygen and nutrients to the cells of the first cell spheroids 91. In this example, the binder material 15 supplies oxygen and nutrients to both the cells of the first cell spheroids 91 and the second cell spheroids 92. In the cell aggregate 90 disposed in the binder material 15 in a state where a plurality of spheroids are scattered in this manner, oxygen and nutrients are also supplied to the cells of the first spheroids 91 and the second spheroids 92. Therefore, the cells are preserved in a viable state.

A (single) cell may be used instead of the cell aggregation such as the cell sheet or the spheroid. In this case, a plurality of cells are scattered in the first membrane surface 15a and the second membrane surface 15b. In the cell aggregate disposed in the binder material 15 in the state where a plurality of cells are scattered in this manner, oxygen and nutrients are also supplied to the cells. Therefore, the cells are preserved in a viable state.

What is claimed is:

1. A composite binder material comprising:
a binder material disposed between a first cell group and a second cell group and formed in a membrane shape; and
a cell non-adhesive portion formed at least in a portion of one membrane surface and/or other membrane surface of the binder material,
wherein the binder material has a honeycomb structure where a plurality of pores open to the one membrane surface are regularly arranged along the one membrane surface,
wherein each of the plurality of pores penetrates in a thickness direction or the plurality of pores penetrate in a surface direction along the surface direction, and
wherein the binder material has a porosity of greater than or equal to 50%.

2. The composite binder material according to claim 1,
wherein the first cell group and the second cell group are any one of a cell aggregation and a plurality of scattered cells.

3. The composite binder material according to claim 2,
wherein the cell aggregation is a cell sheet in which a plurality of cells are aggregated into a sheet shape or a spheroid in which a plurality of cells are aggregated in a spherical shape.

4. The composite binder material according to claim 1,
wherein the plurality of pores penetrate in a thickness direction, and
wherein a thickness of the binder material is within a range of 1 μm to 50 μm.

5. The composite binder material according to claim 1,
wherein the binder material is folded in a state where a first region and a second region of the one membrane surface face each other, and the first cell group is disposed in a state of being caught in the binder material.

6. A cell aggregate comprising:
a first cell group;
a second cell group; and
a composite binder material having a binder material disposed between a first cell group and a second cell group and formed in a membrane shape, and a cell non-adhesive portion formed at least in a portion of one membrane surface and/or other membrane surface of the binder material,
wherein the binder material has a honeycomb structure where a plurality of pores open to the one membrane surface are regularly arranged along the one membrane surface,
wherein each of the plurality of pores penetrates in a thickness direction or the plurality of pores penetrate in a surface direction along the surface direction, and
wherein the binder material has a porosity of greater than or equal to 50%.

7. The cell aggregate according to claim 6,
wherein the binder material is folded in a state where a first region and a second region of the one membrane surface face each other, and the first cell group is caught in the binder material.

8. A cell accumulation method comprising:
a first step of overlapping a first cell group and a composite binder material having a binder material formed in a membrane shape and a cell non-adhesive portion formed at least in a portion of one membrane surface and/or other membrane surface of the binder material each other in a state where the first cell group and the one membrane surface of the binder material or the cell non-adhesive portion formed at least in a portion of the one membrane surface come into contact with each other; and,
a second step of overlapping a second cell group and the composite binder material each other in a state where the second cell group and the other membrane surface of the binder material or the cell non-adhesive portion formed at least in a portion of the other membrane surface come into contact with each other,
wherein the binder material has a honeycomb structure where a plurality of pores open to the one membrane surface are regularly arranged along the one membrane surface,
wherein each of the plurality of pores penetrates in a thickness direction or the plurality of pores penetrate in a surface direction along the surface direction, and
wherein the binder material has a porosity of greater than or equal to 50%.

9. The cell accumulation method according to claim 8,
wherein the plurality of pores in the binder material penetrate in a thickness direction, and
wherein a thickness of the binder material is within a range of 1 μm to 50 μm.

10. The cell accumulation method according to claim 8,
wherein the first cell group and the second cell group are any one of a cell aggregation and a plurality of scattered cells.

11. The cell accumulation method according to claim 10,
wherein the cell aggregation is a cell sheet in which a plurality of cells are aggregated into a sheet shape or a spheroid in which the plurality of cells are aggregated in a spherical shape.

12. The cell accumulation method according to claim 10,
wherein the first cell group is the cell aggregation, and a thickness of the first cell group is less than or equal to 200 μm.

13. The cell accumulation method according to claim 10,
wherein the second cell group is the cell aggregation, and a thickness of the second cell group is less than or equal to 200 μm, and
wherein the plurality of pores in the binder material penetrate in a thickness direction.

14. The cell accumulation method according to claim 8,
wherein the binder material is folded in a state where a first region and a second region of the one membrane surface face each other, and the first cell group is disposed in a state of being caught in the binder material.

15. A cell preservation method for preserving cells by overlapping a first cell group and a second cell group each other, the method comprising:
overlapping a first cell group and a composite binder material having a binder material formed in a membrane shape and a cell non-adhesive portion formed at least in a portion of one membrane surface and/or other membrane surface of the binder material each other in a state where the first cell group and the one membrane surface of the binder material or the cell non-adhesive portion formed at least in a portion of the one membrane surface come into contact with each other;
overlapping a second cell group and the composite binder material each other in a state where the second cell group and the other membrane surface of the binder material or the cell non-adhesive portion formed at least in a portion of the other membrane surface come into contact with each other, and
holding the cell aggregate in which the first cell group and the second cell group overlap each other through the composite binder material in a state of being in contact with a culture solution of the cells,
wherein the binder material has a honeycomb structure where a plurality of pores open to the one membrane surface are regularly arranged along the one membrane surface,
wherein each of the plurality of pores penetrates in a thickness direction or the plurality of pores penetrate in a surface direction along the surface direction, and
wherein the binder material has a porosity of greater than or equal to 50%.

16. The composite binder material according to claim 1,
wherein the cell non-adhesive portion is formed at least in a portion of the one membrane surface and the other membrane surface of the binder material.

17. The composite binder material according to claim 1,
wherein the cell non-adhesive portion is formed to cover the one membrane surface and/or the other membrane surface of the binder material.

18. The composite binder material according to claim 1,
wherein the cell non-adhesive portion is formed to cover the one membrane surface and/or the other membrane surface of the binder material except for a surface opening part.

19. The composite binder material according to claim 1,
wherein the cell non-adhesive portion is formed on the surface of a partition wall between the pores.

20. A method of manufacturing a composite binder disposed between a first cell group and a second cell group and having a binder material formed in a membrane shape and a cell non-adhesive portion, the method comprising:
a first step of preparing a solution by dissolving a material to constitute the cell non-adhesive portion in a solvent; and
a second step of coating the binder material with the solution and drying a coated membrane,
wherein the cell non-adhesive portion is formed at least in a portion of one membrane surface and/or other membrane surface of the binder material,
wherein the binder material has a honeycomb structure where a plurality of pores open to the one membrane surface are regularly arranged along the one membrane surface,
wherein each of the plurality of pores penetrates in a thickness direction or the plurality of pores penetrate in a surface direction along the surface direction, and
wherein the binder material has a porosity of greater than or equal to 50%.

21. The method of manufacturing a composite binder according to claim 20,
wherein in the second step the binder material is coated with the solution by dipping the binder material in the solution.
